(19) United States
(12) Patent Application Publication
(10) Pub. No.: US 2021/0345767 A1
(43) Pub. Date: Nov. 11, 2021

(54) DISINFECTING TOOTHBRUSH BOX

(71) Applicant: SHENZHEN BULUOSEN COSMETICS CO., LTD, SHENZHEN (CN)

(72) Inventor: ZHIMING ZHENG, SHENZHEN (CN)

(21) Appl. No.: 17/239,672

(22) Filed: Apr. 25, 2021

(30) Foreign Application Priority Data
May 9, 2020 (CN) 202010387712.9

(51) Int. Cl. A46B 17/06; A47K 1/09; B65D 35/30; A61L 2/10; A61L 2/26

(52) U.S. Cl. CPC A46B 17/065; A47K 1/09; B65D 35/30; A61L 2/10; A61L 2202/21; A61L 2202/11; A61L 2202/121; A61L 2202/122; A61L 2/26

(57) ABSTRACT

The invention discloses a disinfecting toothbrush box, which comprises a housing, wherein a toothbrush hanging rack space and a toothpaste squeezer space are arranged in the housing; a toothbrush hanging rack is arranged in the toothbrush hanging rack space, a toothpaste squeezer is arranged in the toothpaste squeezer space, an ultraviolet disinfection lamp is arranged in the toothbrush hanging rack space, and the ultraviolet disinfection lamp is arranged at the position capable of irradiating a toothpaste tube opening in the toothpaste squeezer space. The disinfecting toothbrush box has the advantage that the toothpaste tube opening can be sterilized while the toothbrush is disinfected.

### TECHNICAL FIELD

The invention relates to a toothbrush box, in particular to a disinfecting toothbrush box that can sterilize toothbrushes while also sterilizing toothpaste nozzles.

### BACKGROUND TECHNIQUE

Brushing your teeth can remove dental plaque and prevent caries and periodontal disease. Brushing your teeth can also massage your gums, promote blood circulation, and enhance the disease resistance of the gum tissue. Brushing your teeth can clean your mouth. Toothbrushes are used when brushing teeth. After 3 weeks of using toothbrushes, the number of bacteria is as high as one million. If people have a wound in their mouth, these bacteria can easily cause oral diseases. Even if there is no wound, a large number of bacteria are attached to the teeth and mixed with food, and they will enter the body. A toothbrush is parasitic with millions of invisible microorganisms, many of which are harmful to the human body. Especially in a warm and humid bathroom environment, bacteria are more likely to multiply on toothbrushes, which can easily cause oral diseases, such as periodontitis, tooth bleeding, oral odor, and digestive system diseases. Numerous medical studies have shown that oral health is closely related to human health, such as gum problems and heart disease, diabetes, and premature delivery of pregnant women.

In order to keep the toothbrush clean for a long time, some toothbrush sterilizers have appeared now. When in use, people only need to place the toothbrush in the toothbrush sterilizer to sterilize the toothbrush, but everyone's oral cavity is more than one. This is an environment where bacteria exist. If we brush our teeth carefully, we can only reduce the bacteria on the surface of the teeth. It is impossible to completely eliminate them, especially in the gaps between the toothbrush bristles. If multiple people use a tube of toothpaste, everyone's mouth The bacteria in the tube will accumulate in the mouth of the tube, and then be taken away by someone else's toothbrush. The more bacteria accumulate in the mouth of the toothpaste tube, the higher the probability of bacterial transmission. As far as the toothbrush sterilizers appear today, their disinfection effect on toothbrushes is not bad, but they cannot sterilize toothpaste nozzles.

### SUMMARY OF THE INVENTION

In order to overcome the shortcomings of the prior art, the object of the present invention is to provide a disinfecting toothbrush box that can sterilize the toothbrush while also sterilizing the toothpaste nozzle.

The present invention is achieved by the following technical measures. A sterilized toothbrush box includes a housing, a toothbrush hanger space and a toothpaste extruder space are arranged in the housing, and a toothbrush hanger is arranged in the toothbrush hanger space. A toothpaste extruder is arranged in the toothpaste extruder space, an ultraviolet disinfection lamp is arranged in the toothbrush hanger space, and the ultraviolet disinfection lamp is arranged at a position capable of irradiating the toothpaste nozzle in the toothpaste extruder space.

As a preferred mode, the movable cover on the housing is provided with a panel cover capable of covering the front opening of the toothbrush holder.

As a preferred mode, the inner surface of the panel cover is provided with a magnet, and the housing is provided with a magnetic control switch corresponding to the magnet. When the panel cover is on the surface of the housing, the magnetic control switch is connected, and the panel cover leaves the housing. When the surface is on, the magnetic switch is off.

As a preferred mode, a drying device is provided in the housing.

As a preferred manner, a lighting lamp is provided on the surface of the housing, and a human body sensing device on the housing controls the lighting lamp to work when the human body sensing device senses a human body.

As a preferred mode, the housing includes a bottom shell mounted on the wall and a face shell covering the bottom shell, and a toothbrush hanger space for fixing the toothbrush hanger and a toothpaste extruder are provided on the face shell space.

As a preferred mode, a control board is provided in the housing, a power switch button is provided on the housing, and a work indicator light is provided on the power switch button.

As a preferred manner, the toothpaste extruder includes a toothpaste cartridge fixed in a housing, and a silicone sleeve sealed on the head of the toothpaste bottle is sealed on the upper opening of the toothpaste cartridge. There is a crack at the bottom, and the lower opening of the toothpaste tube is set as a one-way valve plate, so that the toothpaste in the toothpaste tube can be pressed down and squeezed out or sucked into the toothpaste tube from the toothpaste bottle under negative pressure; The side of the toothpaste tube is connected with a side tube, and there is a piston in the side tube that seals and reciprocates. The piston reciprocates left and right under the drive of a motor, so that the toothpaste tube is pressed down to squeeze out the toothpaste or is under negative pressure from Inhale toothpaste in the toothpaste bottle.

As a preferred mode, wherein the toothpaste extruder comprises a toothpaste tube fixed in the housing, and the upper opening of the toothpaste tube is sealed with a silicone rubber sleeve on the head of the toothpaste bottle. A sleeve, the bottom of the silicone sleeve is provided with a crack, and the lower opening of the toothpaste cartridge is set as a one-way valve structure, so that the toothpaste in the toothpaste cartridge can be pressed down and squeezed out from the toothpaste bottle under negative pressure The toothpaste is sucked into the toothpaste tube; the side of the toothpaste tube is connected with a side hole, the side hole is sealed with a silicone tube, and the bottom of the silicone tube reciprocates left and right under the drive of a motor. The tube presses down the toothpaste tube to squeeze out the toothpaste or sucks the toothpaste from the toothpaste bottle with negative pressure.

As a preferred mode, a solar cell panel is also provided on the casing.

The ultraviolet disinfection lamp in the present invention is set at a position capable of irradiating the toothpaste tube mouth in the toothpaste extruder space, so that the ultraviolet disinfection lamp can disinfect the toothbrushes in the toothpaste rack space while also disinfecting the toothpaste placed in the toothpaste extruder. The nozzle of the toothpaste is disinfected, thereby avoiding the problem of contamination of the toothbrush after the nozzle of the toothpaste is contaminated.

### DESCRIPTION OF THE DRAWINGS

FIG. 1 is a schematic diagram of the structure of a disinfecting toothbrush box according to an embodiment of the present invention.

FIG. 2 is an exploded view of a disinfecting toothbrush box according to an embodiment of the present invention.

FIG. 3 is another structural schematic diagram of the disinfecting toothbrush box according to the embodiment of the present invention.

FIG. 4 is a schematic structural diagram of a toothpaste extruder for a disinfecting toothbrush box according to an embodiment of the present invention.

FIG. 5 is an exploded view of a toothpaste extruder for a disinfecting toothbrush box according to an embodiment of the present invention.

FIG. 6 is a schematic structural diagram of another toothpaste extruder for a sterilized toothbrush case according to an embodiment of the present invention.

FIG. 7 is an exploded view of another toothpaste extruder of the sterilized toothbrush case according to the embodiment of the present invention.

### DETAILED DESCRIPTION

The present invention will be further described in detail below in conjunction with the embodiments and the accompanying drawings.

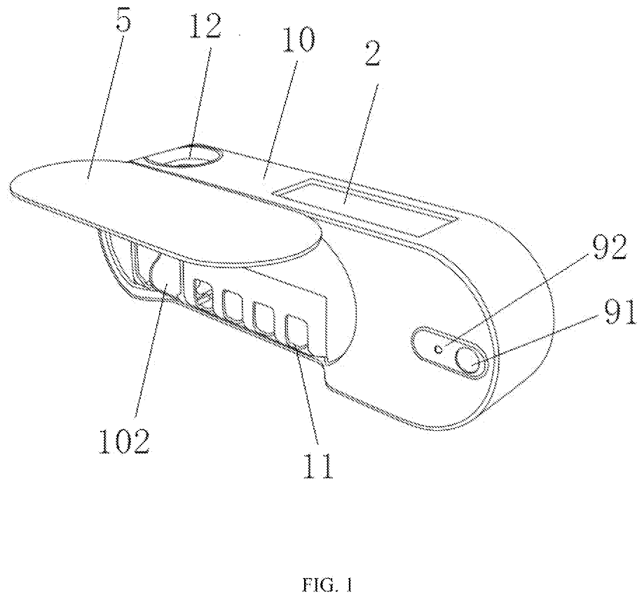

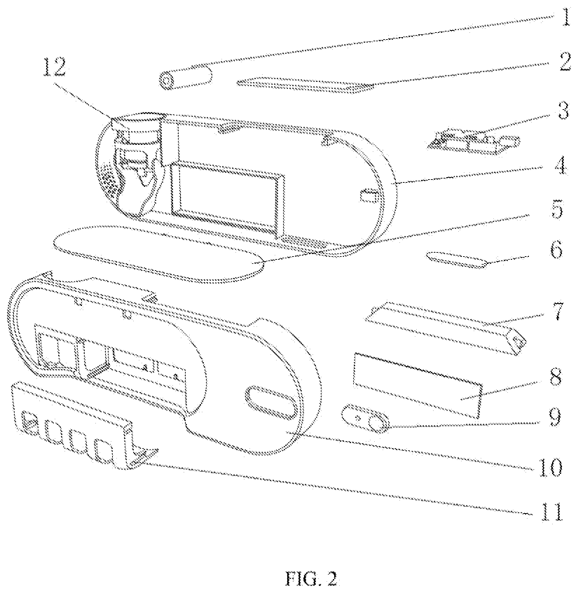

A sterilized toothbrush box, referring to FIGS. 1 to 3, includes a housing in which a toothbrush hanger space 101 and a toothpaste extruder space 102 are arranged, and a toothbrush hanger 11 is arranged in the toothbrush hanger space 101, The toothbrush hanger 11 can be directly integrated with the housing. For the convenience of processing and installation, in this embodiment, the toothbrush hanger 11 is additionally processed and installed on the housing. The toothpaste extruder space 102 is provided with A toothpaste extruder 12, an ultraviolet disinfection lamp 7 is arranged in the toothbrush rack space 102, and the ultraviolet disinfection lamp 7 is arranged at a position capable of irradiating the toothpaste nozzle in the toothpaste extruder space 102, and the two spaces may have holes Physically connected, a translucent plate that can transmit ultraviolet light can also be provided, so that the ultraviolet disinfection lamp 7 disinfects the toothpaste nozzle placed in the toothpaste extruder 12 while disinfecting the toothbrush rack space 101. This avoids the problem of contamination of the toothbrush after the mouth of the toothpaste tube is contaminated.

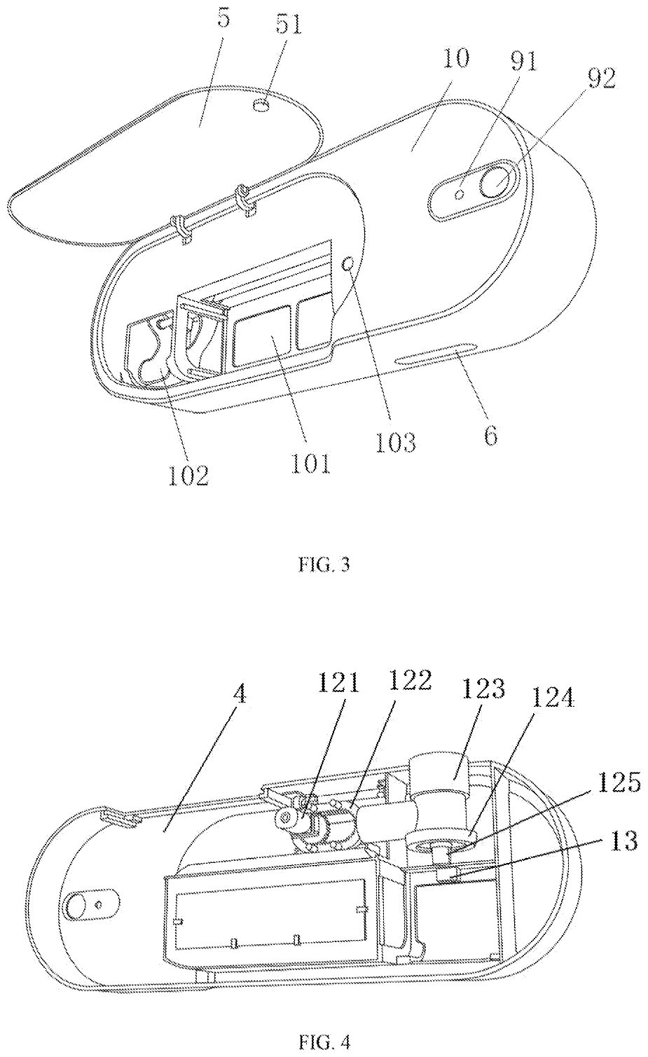

Please refer to FIGS. 1 and 3 for the disinfection toothbrush box of an embodiment. Based on the previous technical solution, it is also possible that the movable cover on the housing is provided with a panel cover 5 capable of covering the front opening of the toothbrush holder 11. The use of an additional panel cover 5. Use the panel cover 5 to shield the ultraviolet disinfection lamp 7 to prevent the ultraviolet light from hurting human eyes.

For the disinfection toothbrush case of an embodiment, please refer to FIGS. 1 and 3. On the basis of the previous technical solution, it can also be specifically that a magnet is provided on the inner surface of the panel cover 5, and the housing is provided with a magnetic control switch 103 corresponding to the magnet. When the panel cover 5 is covered on the surface of the housing, the magnetic control switch 103 is connected, and when the panel cover 5 leaves the surface of the housing, the magnetic control switch 103 is disconnected. The magnetic control switch 103 is used to detect whether the panel cover 5 is closed, so as to prevent the cover from being opened by mistake and hurting people's eyes.

Please refer to FIG. 1 and FIG. 3 for the disinfection toothbrush box of an embodiment. Based on the previous technical solution, a drying device 8 is provided in the housing, and the drying device 8 is used to ensure that the toothbrush in the housing is dry. To avoid bacterial regeneration, in this embodiment, the toothbrush is automatically energized for 15 minutes every 4 hours to ensure that the toothbrush is in a dry state 24 hours a day.

For the disinfection toothbrush box of an embodiment, please refer to FIGS. 1 and 3. On the basis of the previous technical solutions, it can also be specifically that a lighting lamp 6 is provided on the surface of the housing, a human body sensing device 92 on the housing, and When the human body sensing device 92 senses a human body, it controls the lighting lamp to work. The human body sensing device 92 in this embodiment can sense the human body back illuminator 6 (LED light board in this embodiment) within a range of 120° to turn off after being turned on for 30 seconds (this group of functions can also be turned off manually).

For the disinfection toothbrush box of an embodiment, please refer to FIGS. 1 and 3. On the basis of the previous technical solution, the housing may include a bottom shell 4 mounted on the wall and a surface covering the bottom shell 4 The shell 10 is provided with a toothbrush hanger space 101 for fixing the toothbrush hanger and a toothpaste extruder space 102 on the surface shell.

For the disinfection toothbrush box of one embodiment, please refer to FIGS. 1 and 3. On the basis of the previous technical solution, a control board 3 is provided in the housing, and a power switch button 9 is provided on the housing. The power switch button 9 is provided with a work indicator light 91 and a human body sensing device 92.

In another embodiment of the disinfecting toothbrush box, based oil the previous technical solution, a humidity sensor is provided in the housing, and the drying device is stopped when the humidity in the housing is detected to be too low (such as 30% or 40%) 8. When it is detected that the humidity in the housing is too high (such as 70% or 80%), the drying device 8 is started.

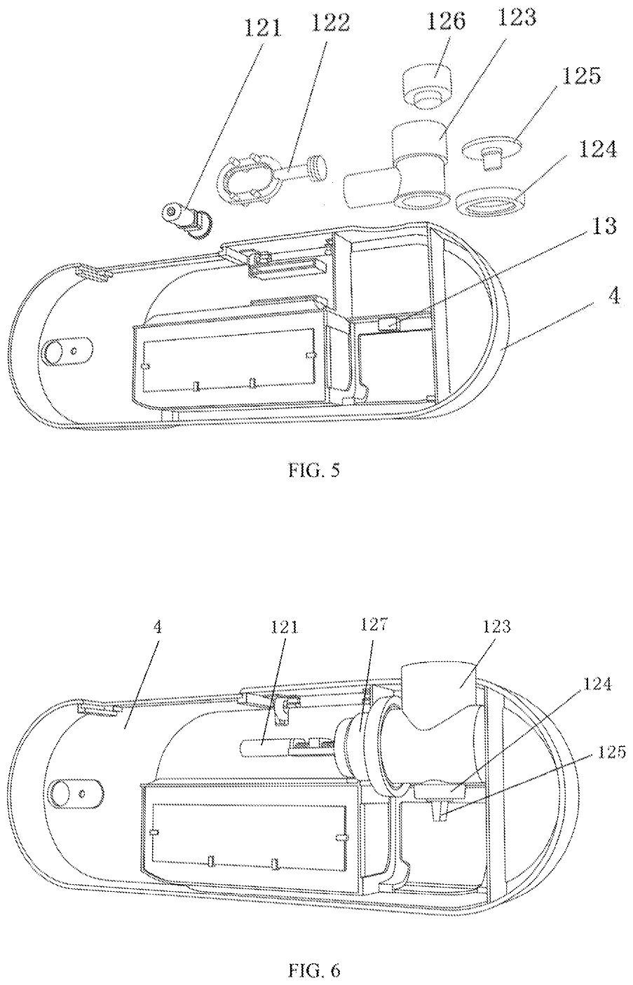

For the disinfection toothbrush box of an embodiment, please refer to FIGS. 4 and 5. Based on the previous technical solution, the toothpaste extruder 12 may include a toothpaste tube 123 fixed in the bottom shell 4. The toothpaste tube The upper opening of 123 is sealed with a silicone sleeve 126 which is sealed and sleeved on the head of the toothpaste bottle. The bottom of the silicone sleeve 126 is provided with a crack. The lower opening of the toothpaste canister 123 is fixed with a one-way valve through a fixing ring 124. The piece 125 enables the toothpaste in the toothpaste tube 123 to be pressed down and squeeze out or suck the toothpaste from the toothpaste bottle into the toothpaste tube 123 under negative pressure; the side of the toothpaste tube 123 is connected with a side tube, the side tube There is a piston 122 in the inner seal that reciprocates, and the piston 122 reciprocates left and right under the drive of a motor 121, so that the toothpaste tube 123 is pressed down to squeeze out the toothpaste or sucked into the toothpaste bottle under negative pressure; the toothpaste tube An infrared detection device 13 is also arranged near the lower opening of 123. When the infrared detection device 13 detects that a toothbrush extends into the lower opening of the toothpaste tube 123, the motor 121 is controlled to work to squeeze toothpaste from the toothpaste tube 123 onto the toothbrush.

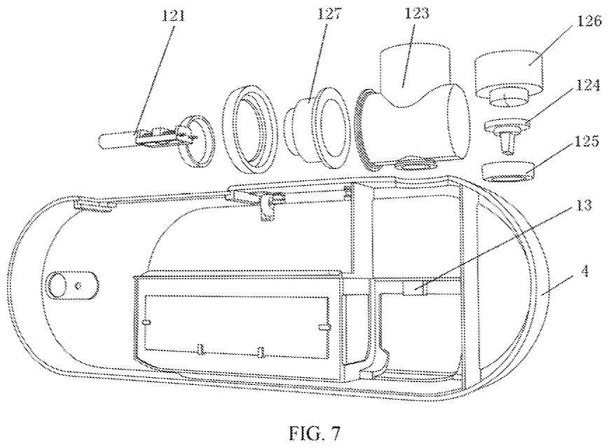

In another embodiment of the sterilized toothbrush box, please refer to FIGS. 6 and 7. On the basis of the previous technical solutions, the toothpaste extruder 12 may include a toothpaste tube 123 fixed in the bottom shell 4. The toothpaste The upper opening of the barrel 123 is sealed with a silicone sleeve 126 that is sealed and sleeved on the head of the toothpaste bottle. The bottom of the silicone sleeve 126 is provided with a crack. The lower opening of the toothpaste barrel 123 is fixed with a one-way through a fixing ring 124. The valve plate 125 enables the toothpaste in the toothpaste tube 126 to be pressed down and squeezed out or under negative pressure to suck the toothpaste from the toothpaste bottle into the toothpaste tube 126; the side of the toothpaste tube 126 is connected with a side hole. The sealing cover on the side hole is provided with a silicone tube 127. The bottom of the silicone tube 127 reciprocates left and right under the drive of a motor 121. The silicone tube 127 presses the toothpaste tube 123 to squeeze out the toothpaste or draw negative pressure from it. A toothpaste bottle is sucked into the toothpaste bottle; an infrared detection device 13 is also provided near the lower opening of the toothpaste tube 123. When the infrared detection device 13 detects that a toothbrush is inserted into the lower opening of the toothpaste tube 123, the motor 121 is controlled to work to make the toothpaste from Squeeze the toothpaste tube 123 onto the toothbrush.

Please refer to FIG. 1 and FIG. 3 for the disinfecting toothbrush box of an embodiment. Based on the previous technical solution, it can also be specifically that a rechargeable battery 1 is provided in the housing, and the rechargeable battery 1 is used for power supply.

Please refer to FIG. 1 and FIG. 3 for the disinfection toothbrush box of an embodiment. Based on the previous technical solution, it can also be specifically provided with a solar panel 2 on the housing. The solar panel 2 can be used to convert the environment Light energy supplies power to the device. In one embodiment, a set of solar panel charging circuits are used, and the main board is powered by an 18650 battery. When powered by a USB cable, it can work normally without a lithium battery.

The above is the description of the disinfection toothbrush box of the present invention to help understand the present invention, but the embodiments of the present invention are not limited by the above-mentioned examples, and any changes, modifications, substitutions, combinations, and combinations made without departing from the principles of the present invention Simplified, all should be equivalent replacement methods, and all are included in the protection scope of the present invention.

What is claimed is:

1. A disinfecting toothbrush box, characterized in that it comprises a housing, a toothbrush hanger space and a toothpaste extruder space are arranged in the housing, a toothbrush hanger is arranged in the toothbrush hanger space, and the toothpaste extruder A toothpaste extruder is arranged in the space, an ultraviolet disinfection lamp is arranged in the toothbrush rack space, and the ultraviolet disinfection lamp is arranged at a position capable of irradiating the toothpaste nozzle in the toothpaste extruder space.

2. The disinfecting toothbrush box according to claim 1, wherein the movable cover on the housing is provided with a panel cover capable of covering the front opening of the toothbrush hanger.

3. The disinfecting toothbrush box according to claim 2, wherein the inner surface of the panel cover is provided with a magnet, the housing is provided with a magnetic control switch corresponding to the magnet, and the magnetic control switch is provided when the panel cover is on the surface of the housing. When the panel cover leaves the surface of the housing, the magnetic control switch is disconnected.

4. The disinfecting toothbrush box according to claim 1, wherein a drying device is provided in the housing.

5. The disinfecting toothbrush box according to claim 1, wherein the surface of the housing is provided with an illuminating lamp, and a human body sensing device on the housing is configured to control the operation of the illuminating lamp when the human body sensing device senses a human body.

6. The disinfecting toothbrush box according to claim 1, wherein the housing includes a bottom shell mounted on the wall and a face shell covering the bottom shell, and the face shell is provided with a toothbrush holder. Toothbrush hanger space and toothpaste extruder space.

7. The disinfecting toothbrush box according to claim 1, wherein a control panel is provided in the housing, a power switch button is provided on the housing, and a work indicator is provided on the power switch button.

8. The disinfecting toothbrush box according to claim 1, wherein the toothpaste extruder comprises a toothpaste tube fixed in the housing, and the upper opening of the toothpaste tube is sealed with a silicone rubber sleeve on the head of the toothpaste bottle. A sleeve, the bottom of the silicone sleeve is provided with a crack, and the lower opening of the toothpaste tube is set as a one-way valve plate, so that the toothpaste in the toothpaste tube can be pressed down and squeezed out from the toothpaste bottle under negative pressure The toothpaste is sucked into the toothpaste tube; the side of the toothpaste tube is connected with a side tube, the side tube is sealed with a piston that reciprocates, and the piston reciprocates left and right under the drive of a motor, so that the toothpaste tube is compressed Squeeze out the toothpaste or suck the toothpaste from the toothpaste bottle under negative pressure.

9. The disinfecting toothbrush box according to claim 1, wherein the toothpaste extruder comprises a toothpaste tube fixed in the housing, and the upper opening of the toothpaste tube is sealed with a silicone rubber sleeve on the head of the toothpaste bottle. A sleeve, the bottom of the silicone sleeve is provided with a crack, and the lower opening of the toothpaste cartridge is set as a one-way valve structure, so that the toothpaste in the toothpaste cartridge can be pressed down and squeezed out from the toothpaste bottle under negative pressure The toothpaste is sucked into the toothpaste tube; the side of the toothpaste tube is connected with a side hole, the side hole is sealed with a silicone tube, and the bottom of the silicone tube reciprocates left and right under the drive of a motor. The tube presses down the toothpaste tube to squeeze out the toothpaste or sucks the toothpaste from the toothpaste bottle with negative pressure.

10. The disinfecting toothbrush box according to claim 1, characterized in that: a solar cell panel is also provided on the casing.
